(19) United States
(12) Patent Application Publication
(10) Pub. No.: US 2026/0139314 A1
(43) Pub. Date: May 21, 2026

(54) COMPOSITION AND METHOD FOR PREDICTING SULFONYLUREA DEPENDENCY USING GIPR MARKER

(71) Applicant: Seoul National University Hospital, Seoul (KR)

(72) Inventor: Hye Seung Jung, Seoul (KR)

(21) Appl. No.: 19/119,764

(22) PCT Filed: Sep. 27, 2023
(86) PCT No.: PCT/KR2023/015061

§ 371 Date: Apr. 10, 2025

(30) Foreign Application Priority Data
Oct. 12, 2022 (KR) 10-2022-0130239
Aug. 14, 2023 (KR) 10-2023-0106511

(51) Int. Cl. C12Q 1/6883

(52) U.S. Cl. CPC C12Q 1/6883; C12Q 2600/106; C12Q 2600/156

(57) ABSTRACT

The present invention relates to a composition and method for predicting sulfonylurea dependency in diabetes mellitus patients by identifying the mRNA level of GIPR (gastric inhibitory polypeptide receptor), the level or activity of GIPR protein, or mutations inhibiting the level or activity of GIPR, and for modulating such dependency by regulating these factors.

### TECHNICAL FIELD

The present invention relates to a composition for predicting sulfonylurea dependency and a method for predicting sulfonylurea dependency.

### BACKGROUND

Compared to research on markers related to susceptibility to diabetes mellitus onset, studies on markers related to the effects of diabetes mellitus treatments are minimal. As a result, there are no competing technologies for genetic markers with clinical significance concerning the use of diabetes mellitus treatments, and prescriptions are currently based on the experience of medical professionals.

Currently, nine classes of medications are used for diabetes mellitus treatment, and prescription guidelines by most related societies are not based on predictions of blood glucose-lowering effects or do not contribute to such predictions (Standards of Medical Care in Diabetes, Diabetes Care January 2022; Korean Diabetes Association Diabetes Clinical Practice Guidelines 2021).

Sulfonylureas are a class of highly potent blood glucose-lowering agents with the longest history of use. However, compared to other classes of medications, they are associated with a higher risk of hypoglycemia. Furthermore, the development of other oral blood glucose-lowering agents with proven cardiovascular protective effects has resulted in a decline in the overall prescription priority of sulfonylureas. Nonetheless, it has been observed that certain patient groups heavily rely on sulfonylureas for blood glucose control. For these specific subset of patients, the preferential use of sulfonylureas is necessary, but no clinical characteristics or markers predicting such dependency have been identified to date.

Therefore, there is a need to discover markers related to sulfonylurea dependency for diabetes mellitus treatment and to develop technologies that can identify sensitivity to sulfonylureas based on these markers.

### DETAILED DESCRIPTION OF THE INVENTION

### Technical Problem

The present invention aims to identify genes that determine sulfonylurea dependency and to provide a composition for predicting sulfonylurea dependency.

The present invention aims to provide a method for offering information to predict sulfonylurea dependency.

### Technical Solution

1. A composition for predicting sulfonylurea dependency in diabetes mellitus patients, comprising an agent that identifies the mRNA level of GIPR (gastric inhibitory polypeptide receptor), the level or activity of GIPR protein, or mutations inhibiting the level or activity of GIPR.

2. The composition for predicting sulfonylurea dependency in diabetes mellitus patients according to item 1, wherein the mutations are missense mutations, frameshift mutations, nonsense mutations, or splice site mutations.

3. The composition for predicting sulfonylurea dependency in diabetes mellitus patients according to item 1, wherein the agent is a primer or probe that specifically binds to the GIPR gene.

4. The composition for predicting sulfonylurea dependency in diabetes mellitus patients according to item 1, wherein the mutations are one or more single nucleotide variations (SNVs) selected from the group consisting of rs550405192, rs13306403, rs13306402, rs554179666, rs1194979043, rs149510000, rs759654048, rs764005735, rs935395843, rs755629061, rs749728382, rs779198689, rs1271638992, rs775963892, rs778756249, rs146268621, rs753645152, rs771165150, rs771830344, rs144328094, rs1159478274, rs1292381802, rs550405192, rs747395645, rs183524419, and rs757704281.

5. A composition for modulating sulfonylurea dependency in diabetes mellitus patients, comprising an agent that regulates the mRNA level of GIPR (gastric inhibitory polypeptide receptor), the level or activity of GIPR protein, or induces mutations that inhibit the level or activity of GIPR.

6. The composition for modulating sulfonylurea dependency in diabetes mellitus patients according to item 5, wherein the mutations in GIPR are one or more single nucleotide variations (SNVs) selected from the group consisting of rs550405192, rs13306403, rs13306402, rs554179666, rs1194979043, rs149510000, rs759654048, rs764005735, rs935395843, rs755629061, rs749728382, rs779198689, rs1271638992, rs775963892, rs778756249, rs146268621, rs753645152, rs771165150, rs771830344, rs144328094, rs1159478274, rs1292381802, rs550405192, rs747395645, rs1183524419, and rs757704281.

7. A method for providing information to predict sulfonylurea dependency in diabetes mellitus patients, comprising the step of identifying the mRNA level of GIPR (gastric inhibitory polypeptide receptor), the level or activity of GIPR protein, or mutations in GIPR from a biological sample isolated from an individual.

8. The method for providing information to predict sulfonylurea dependency in diabetes mellitus patients according to item 7, wherein the mutations are missense mutations, frameshift mutations, nonsense mutations, or splice site mutations.

9. The method for providing information to predict sulfonylurea dependency in diabetes mellitus patients according to item 7, wherein the agent is a primer or probe that specifically binds to the GIPR gene.

10. The method for providing information to predict sulfonylurea dependency in diabetes mellitus patients according to item 7, wherein the mutations in GIPR are one or more single nucleotide variations (SNVs) selected from the group consisting of rs550405192, rs13306403, rs13306402, rs554179666, rs1194979043, rs149510000, rs759654048, rs764005735, rs935395843, rs755629061, rs749728382, rs779198689, rs1271638992, rs775963892, rs778756249, rs146268621, rs753645152, rs771165150, rs771830344, rs144328094, rs1159478274, rs1292381802, rs550405192, rs747395645, rs1183524419, and rs757704281.

11. The method for providing information to predict sulfonylurea dependency in diabetes mellitus patients according to item 7, further comprising the step of providing information that an individual with the mutations has sulfonylurea dependency.

12. The method for providing information to predict sulfonylurea dependency in diabetes mellitus patients according to item 7, further comprising the step of providing information that if the level or activity is lower than that of a control group, the individual is more likely to have sulfonylurea dependency compared to the control group.

### Effect of the Invention

The present invention can be used to predict whether a diabetes mellitus patient has excellent blood glucose-lowering effects from sulfonylureas by using GIPR (gastric inhibitory polypeptide receptor) as a marker and to provide information for this purpose.

### BRIEF DESCRIPTION OF THE DRAWINGS

FIG. 1 illustrates a schematic method for deriving sulfonylurea dependency-related variants according to the present invention.

DM, diabetes mellitus; FPG, fasting plasma glucose; MAF, minor allele frequency; SNV, single nucleotide variant; SU, sulfonylurea; WES, whole exome sequencing

FIG. 2 shows the blood levels of HbA1c (glycated hemoglobin) or FPG (fasting plasma glucose) after discontinuation of sulfonylurea administration.

Values are expressed as mean±standard deviation.
**, p<0.001 between the two groups by two-way repeated measures ANOVA
FPG, fasting plasma glucose; peak, highest measures within 6 months after SU discontinuation; SU, sulfonylureas

FIG. 3 illustrates the level of GIPR expression and the degree of insulin secretion induced by GIP stimulation following siGipr treatment.

INS1 rat insulinoma cells were transfected with 80-nM siRNA for Gipr and negative control.

(a) Quantitative RT-PCR: Gipr mRNA normalized to 18s expression was downregulated by siGipr.

(b) Western blot: GIPR protein levels normalized to tubulin expression were also reduced by siGipr.

(c) ELISA: Insulin secretion induced by GIP treatment (50 nM for 1 hour) was attenuated by siGipr.
siGipr, siRNA for Gipr; siNS, siRNA for negative control; siRNA, small interfering RNA; NS, no significant difference

FIG. 4 shows the changes in insulin secretion induced by low-dose SU stimulation in insulinoma cells with GIPR expression suppressed.

INS1 rat insulinoma cells were transfected with siGipr and siNS (80 nM) and stimulated for insulin secretion with low-dose SU (50 nM glimepiride for 30 minutes). Insulin secretion was measured using an ELISA kit.

(a) Physiological condition (b) Diabetic condition simulated by pre-treating with 15-mM glucose and 150-μM palmitic acid for 24 hours (glucolipotoxicity).

Values are expressed as mean±standard deviation.

*, p<0.05 by two-way ANOVA

FIG. 5 shows the intracellular and extracellular ATP levels in insulinoma cells with GIPR expression suppressed.

INS1 cells were transfected with siGipr and siNS, and ATP levels in cell lysates (a) and media (b) were measured under glucolipotoxic and non-glucolipotoxic conditions for 24 hours.

*, p<0.05 by two-way ANOVA

FIG. 6 shows the intracellular Vdac1 mRNA levels in insulinoma cells with GIPR expression suppressed.

INS1 cells were transfected with siGipr and siNS, and Vdac1 mRNA levels were normalized to 18s expression and compared under glucolipotoxic and non-glucolipotoxic conditions for 24 hours.

*, p<0.05, by Kruskal-Wallis test

FIG. 7 shows the changes in insulin secretion induced by low-dose SU stimulation in insulinoma cells with GIPR function suppressed.

INS1 rat insulinoma cells were treated with a GIPR inhibitor (rat GIP(3-30), 100 nM, 24 h) and the inhibition of insulin secretion by GIP was confirmed. Insulin secretion was stimulated with low-dose SU (50 nM glimepiride for 30 minutes) and measured using an ELISA kit.

### FORM FOR PRACTICING THE INVENTION

Hereinafter, the present invention will be described in detail. Unless otherwise defined, all terms in this specification have the same general meanings understood by those skilled in the art to which the present invention pertains. In cases where the meaning of a term used in this specification conflicts with its general meaning, the meaning as used in this specification shall prevail.

The present invention relates to a composition for predicting sulfonylurea dependency in diabetes mellitus patients, comprising an agent that identifies the mRNA level of GIPR (gastric inhibitory polypeptide receptor), the level or activity of the GIPR protein, or mutations inhibiting the level or activity of GIPR.

In the present invention, “sulfonylurea dependency” refers to the extent to which a diabetes mellitus patient must rely exclusively on sulfonylureas or prioritize sulfonylureas over other medications for blood glucose lowering. Specifically, it means that blood glucose lowering is either not observed or does not meet expectations with other conventionally known blood glucose-lowering agents, such as metformin, gliptin, or pioglitazone, but shows the predicted blood glucose effect only when sulfonylureas are administered, necessitating the prioritized use of sulfonylureas. The sulfonylurea dependency prediction composition of the present invention is a composition that can predict whether a diabetes mellitus patient has sulfonylurea dependency based on the agents that identify the mRNA level of GIPR, the level or activity of the GIPR protein, or mutations inhibiting the level or activity of GIPR. Specifically, in cases where the mRNA or protein level of GIPR is low, the activity is reduced, or mutations that inhibit its function, activity, or level are present, it can be predicted that the patient has high sulfonylurea dependency. However, this is not limited thereto.

In the present invention, mutations in the gene are included within the scope of the present invention without limitation, provided they involve additions, deletions, or changes in the nucleotide sequence of the gene. For example, the mutations may include missense mutations, frameshift mutations, nonsense mutations, or splice site mutations, preferably those that induce mutations affecting the functional protein of the gene. However, this is not limited thereto.

The GIPR gene, mRNA, and protein may have sequences specific to the prediction target. The sequences for each species are all known. For example, in humans, the gene may correspond to Gene ID: 2696, the mRNA may correspond to the sequence of SEQ ID No. 1, and the protein may correspond to the sequence of SEQ ID No. 2.

The mutations in the GIPR gene of the present invention may include those that render the GIPR gene nonfunctional or reduce its role. These mutations are not limited but may include one or more single nucleotide variations (SNVs) selected from the group consisting of rs550405192, rs13306403, rs13306402, rs554179666, rs1194979043, rs149510000, rs759654048, rs764005735, rs935395843, rs755629061, rs749728382, rs779198689, rs1271638992, rs775963892, rs778756249, rs146268621, rs753645152, rs771165150, rs771830344, rs144328094, rs1159478274, rs1292381802, rs550405192, rs747395645, rs1183524419, and rs757704281. However, this is not limited thereto.

GIPR: Variants derived from SU-dependent patients, or stop-gained variants.

TABLE 1

| | Position | | |
|---|---|---|---|
| rsID | (GRCh37/hq19) | Reference | Alternate |
| rs13306403 | 19-46181186 | G | T |
| rs13306402 | 19-46177353 | C | T |
| rs550405192 | 19-46180630 | C | A |
| rs554179666 | 19-46172839 | C | T |
| rs1194979043 | 19-46173908 | C | T |
| rs149510000 | 19-46173929 | C | T |
| rs759654048 | 19-46173937 | G | A |
| rs764005735 | 19-46173938 | G | T |
| rs935395843 | 19-46174583 | G | A |
| rs755629061 | 19-46174622 | C | A |
| rs749728382 | 19-46174628 | G | A |
| rs779198689 | 19-46174640 | G | A |
| rs1271638992 | 19-46177357 | T | A |
| rs775963892 | 19-46177429 | T | A |
| rs778756249 | 19-46177998 | C | T |
| rs146268621 | 19-46178019 | C | T |
| rs753645152 | 19-46178037 | C | T |
| rs771165150 | 19-46178077 | G | A |
| rs771830344 | 19-46180272 | G | A |
| rs144328094 | 19-46180293 | C | G |
| rs1159478274 | 19-46180330 | G | T |
| rs1292381802 | 19-46180624 | C | G |
| rs550405192 | 19-46180630 | C | G |
| rs747395645 | 19-46181003 | G | A |
| rs1183524419 | 19-46181224 | C | T |
| rs757704281 | 19-46181416 | G | T |

In the present invention, the agents for identifying the levels of mRNA or protein, protein activity, or mutations can be used without limitation as long as they can confirm DNA, mRNA sequences, protein sequences, protein activity, etc. For example, they may include primers, probes, antibodies, or antisense nucleic acids, but are not limited thereto. Using the primers, probes, or antisense nucleic acids, nucleic acid sequences with specific alleles at the mutation sites can be amplified or their presence can be identified, and the quantity of proteins encoded by the gene can also be measured using the antibodies.

In the present invention, diabetic patients are included within the scope of the invention regardless of the type of diabetes. Preferably, it may be Type 2 diabetes mellitus (T2DM), but it is not limited thereto.

Additionally, the present invention relates to a composition for modulating sulfonylurea (SU) dependency in diabetic patients, comprising an agent that regulates the mRNA level of GIPR (gastric inhibitory polypeptide receptor), the level or activity of GIPR protein, or induces mutations inhibiting the level or activity of GIPR.

As described above, “sulfonylurea (SU) dependency” refers to the extent to which a diabetic patient must rely exclusively on sulfonylureas or prioritize sulfonylureas over other medications for blood glucose lowering. The GIPR mRNA level; the level or activity of the GIPR protein; and mutations in GIPR are associated with SU dependency, and modulating these can regulate SU dependency. For example, SU dependency may increase by suppressing the GIPR mRNA level, the level or activity of the GIPR protein, or by inducing mutations.

In the present invention, mutations in the GIPR gene are included within the scope of the invention without limitation, as long as they impair or reduce the function of the GIPR gene. For example, such mutations may include rs550405192, rs13306403, rs13306402, rs554179666, rs1194979043, rs149510000, rs759654048, rs764005735, rs935395843, rs755629061, rs749728382, rs779198689, rs1271638992, rs775963892, rs778756249, rs146268621, rs753645152, rs771165150, rs771830344, rs144328094, rs1159478274, rs1292381802, rs550405192, rs747395645, rs1183524419, and rs757704281, but are not limited thereto.

In the present invention, agents that regulate the mRNA level of GIPR (gastric inhibitory polypeptide receptor), the level or activity of the GIPR protein, or induce mutations that inhibit the level or activity of GIPR can be used without limitation, provided they perform these roles. For example, such agents may include antibodies, antisense nucleic acids, natural products, or compounds, but are not limited thereto.

Additionally, the present invention relates to a method for providing information for predicting sulfonylurea dependency in diabetic patients, comprising a step of identifying the mRNA level of GIPR (gastric inhibitory polypeptide receptor), the level or activity of the GIPR protein, or mutations inhibiting the level or activity of GIPR from a biological sample isolated from an individual.

In the present invention, the method for providing information for predicting sulfonylurea dependency in diabetic patients may further comprise a step of amplifying and sequencing DNA from a biological sample isolated from an individual to obtain the individual's genotype information. The methods for DNA amplification, sequencing analysis, and obtaining genotype information can be used without limitation as long as they are known to those skilled in the art.

Additionally, the method for providing information may further comprise a step of providing information that an individual with the mutations has sulfonylurea dependency. Specifically, individuals with the aforementioned mutations, more specifically rs550405192, rs13306403, rs13306402, rs554179666, rs1194979043, rs149510000, rs759654048, rs764005735, rs935395843, rs755629061, rs749728382, rs779198689, rs1271638992, rs775963892, rs778756249, rs146268621, rs753645152, rs771165150, rs771830344, rs144328094, rs1159478274, rs1292381802, rs550405192, rs747395645, rs1183524419, and rs757704281, may be provided with information indicating that they have sulfonylurea dependency. However, this is not limited thereto.

Additionally, the method may further comprise a step of providing information that when the levels are lower than those of a control group, the individual is more likely to have sulfonylurea dependency compared to the control group.

In the present invention, the control group refers to diabetic patients whose blood glucose is lowered by other blood glucose-lowering agents, such as metformin, gliptin, and pioglitazone, rather than sulfonylureas, or to non-diabetic individuals. The control group may consist of a single individual or a group of two or more individuals. If the control group consists of two or more individuals, the levels of the control group may refer to the mean, median, or other statistics of the levels of the individual members of the group. Therefore, diabetic patients with levels lower than those of the control group may be provided with information that they are relatively more likely to have sulfonylurea dependency compared to the control group.

Hereinafter, the present invention will be described in detail by presenting specific examples.

### Experimental Example 1. Identification of SU-Dependent Patients Through Retrospective Medical Record Review

A retrospective observational study was conducted on adult patients with type 2 diabetes mellitus (T2DM) who visited Seoul National University Hospital between 2009 and 2015. These patients had been taking low-dose sulfonylureas (SU, glimepiride equivalent ≥2 mg/day) and discontinued their use.

SU dependency was defined as meeting all the following criteria:

(1) Patients with HbA1c≤7.0% or HbA1c≥7.5% (in the case of hypoglycemic risk) for at least six months while taking low-dose SU (glimepiride equivalent 2 mg/day).

(2) Patients who showed an increase in HbA1c≥1.2% within three months or an increase in HbA1c≥1.5% within six months after discontinuing SU, regardless of the addition or administration of other oral diabetes medications.

(3) Patients who resumed SU administration.

(4) Patients who exhibited a decrease in HbA1c≥0.8% or a reduction in fasting blood glucose ≥40 mg/dL within three months of resuming SU.

Patients were considered SU-independent if their HbA1c remained below 7.5% after discontinuing SU, and SU was not resumed until the final data collection in 2019. The SU-independent control group was selected from T2DM patients with a disease duration of over 10 years and was matched with SU-dependent patients in terms of age, sex, and anti-diabetic medications.

Patients were excluded from analysis if they had impaired renal function (serum creatinine >1.4 mg/dL or estimated glomerular filtration rate [eGFR]<50 mL/min/1.73 m²), clinically significant liver disease, were taking medications such as glucocorticoids that could affect glycemic control, or experienced serious medical issues during changes in SU use.

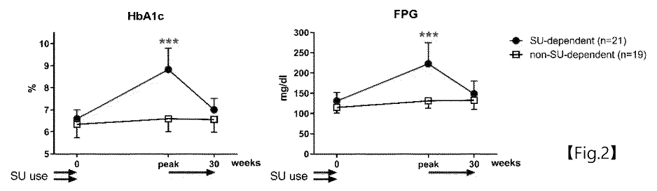

As a result, 21 SU-dependent patients and 19 SU-independent control patients were selected. SU-dependent patients showed significant increases in blood glucose levels after SU discontinuation (HbA1c increased from 6.6±0.4% to 8.8±1.0% at 20 weeks) and significant reductions in blood glucose after resuming SU (HbA1c decreased to 6.9±0.5% at 12 weeks), as well as similar trends in fasting blood glucose. This indicated a significantly distinct drug response compared to the SU-independent control group (FIG. 2). However, clinical characteristics and anti-diabetic medication use before SU discontinuation were similar in both groups (Table 2), suggesting difficulty in distinguishing SU responsiveness prior to discontinuation.

Comparisons of Clinical Characteristics and Anti-diabetic Medications Before and After SU Discontinuation

TABLE 2

| | | SU-dependent | Non-SU-dependent | |
|---|---|---|---|---|
| | | (n = 21) | (n = 19) | P* |
| Male (%) | | 62 | 58 | 1.000 |
| Age (years) | | 66.1 ± 9.9 | 66.3 ± 7.5 | 0.966 |
| Duration of diabetes (years) | | 16.0 (5~36) | 16.7 ± 3.9 | 0.291 |
| Body mass index (kg/m²) | | 24.7 ± 3.0 | 25.5 ± 4.6 | 0.524 |
| T2DM in the 1^{st}-degree family (%) | | 81 | 75 | 0.705 |
| Fasting C-peptide (nmol/L) | | 0.99 ± 0.57 | 0.94 ± 0.44 | 0.873 |
| | | (n = 11) | (n = 8) | |
| Dyslipidemia^{†} (%) | | 90 | 95 | 1.000 |
| Hypertension (%) | | 90 | 89 | 1.000 |
| Ischemic heart diseases (%) | | 19 | 11 | 0.381 |
| Ischemic stroke (%) | | 19 | 11 | 0.381 |
| Urine albumin/creatinine ratio (mg/g) | | 12 (4~127) | 23 (0~97) | 0.154 |
| eGFR (mL/min/1.73 m²) | | 80.5 ± 18.7 | 82.2 ± 18.0 | 0.775 |
| Retinopathy (%) | | 48 | 26 | 0.144 |
| SU dose (mg/day as glimepiride- | | 0.90 ± 0.36 | 0.95 ± 1.05 | 0.832 |
| equivalent [2,3]) | | | | |
| Anti-diabetic agents | Metformin | 86 | 95 | 0.607 |
| before | Gliptins | 38 | 32 | 0.748 |
| SU discontinuation (%) | Pioglitazone | 19 | 21 | 1.000 |
| Anti-diabetic agents | Metformin | 90 | 100 | 1.000 |
| during | Gliptins | 62 | 68 | 0.748 |
| SU discontinuation (%) | Pioglitazone | 29 | 21 | 0.721 |
| Anti-diabetic agents | Metformin | 90 | Not applicable | |
| after | Gliptins | 43 | | |
| SU resumption (%) | Pioglitazone | 19 | | |

Data are expressed as mean±standard deviation or median (range).
eGFR: estimated glomerular filtration rate; SU: sulfonylureas; T2DM: type 2 diabetes mellitus.
*Analyzed by Student's t-test, Mann-Whitney test, or Fisher's exact test.

### Experimental Example 2. Whole-Exome Sequencing (WES)

To analyze the genetic characteristics of SU-dependent patients, whole-exome sequencing was planned to identify rare variants with large effect sizes due to the limited sample size. Blood samples for DNA extraction were collected from 17 of the 21 SU-dependent patients who voluntarily submitted written consent.

The study was conducted in accordance with the Declaration of Helsinki and Good Clinical Practice principles, and was approved by the Institutional Review Board (#1407-103-596). Exome sequencing was performed by Macrogen (Seoul, Korea). Briefly, DNA was extracted from blood leukocytes, exome capturing was performed using SureSelect v4+UTR (Agilent Technologies, Santa Clara, CA, USA), and sequencing was carried out on a HiSeq 2000 sequencing system (Illumina Inc., San Diego, CA, USA) with 100-fold coverage. Sequence reads were aligned to the UCSC genome assembly hg19 using BWA, and variants were called and annotated using SAMtools and ANNOVAR software.

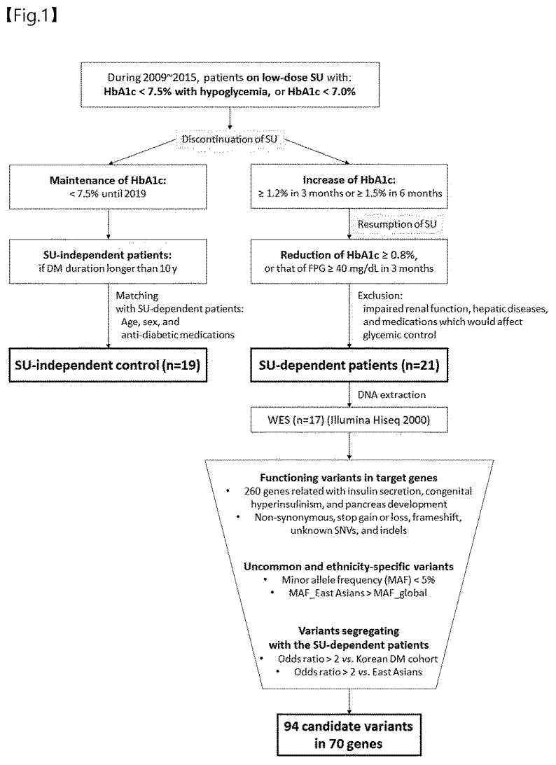

### Experimental Example 3. Selection of Candidate Variants (FIG. 1)

To conduct gene-based analysis, 260 target genes associated with beta-cell function and mass were selected. Single nucleotide variants (SNVs) altering amino acid sequences were screened from these genes based on the whole-exome sequencing results. Since type 2 diabetes is a complex disease influenced significantly by rare variants (Am J Hum Genet. 2001; 69:124), and considering the small sample size of this study, the analysis was limited to uncommon variants (minor allele frequency <5%, 1000 Genomes Phase 1).

It is known that variations in drug-response genes exhibit significant differences among ethnic groups (Genome Med. 2017; 9(1):117). The high usage of SU among East Asians (EAS) suggests good SU response in this population (Diabetes Obes Metab. 2023 January; 25(1):208). Therefore, variants with higher minor allele frequencies in East Asians (EAS) compared to the global average frequency were selected (Genome Aggregation Database [gnomAD], v2.1.1).

To further select variants enriched in SU-dependent patients, variants with odds ratios ≥2 were identified by comparison with two control groups: East Asians (gnomAD_EAS) or the Korean T2DM cohort (SNUH Project, http://koex.snu.ac.kr). As a result, 94 single nucleotide variants (SNVs) in 70 genes were selected as candidate SU dependency-related variants (Table 3).

TABLE 3
Rare/Amino acid-changing/East Asian-enriched/SU-
dependent patient-focused SNV candidates

| | |
|---|---|
| 1 | ABCC8:NM_000352:exon12:c.G1678A:p.V560M, |
| 2 | ACLY:NM_198830:exon18:c.C2093T:p.P698L, |
| | ACLY:NM_001096:exon19:c.C2123T:p.P708L, |
| 3 | AKAP5:NM_004857:exon2:c.G871A:p.G291R, |
| 4 | APOA5:NM_052968:exon4:c.G538C:p.V180L, |
| | APOA5:NM_001166598:exon4:c.G538C:p.V180L, |
| 5 | APOA5:NM_052968:exon4:c.G553T:p.G185C, |
| | APOA5:NM_001166598:exon4:c.G553T:p.G185C, |
| 6 | ARAP1:NM_001040118:exon18:c.G2518A:p.E840K, |
| | ARAP1:NM_015242:exon16:c.G1783A:p.E595K, |
| | ARAP1:NM_001135190:exon15:c.G1600A:p.E534K, |
| 7 | ARAP1:NM_001040118:exon7:c.G883A:p.G295R, |
| | ARAP1:NM_015242:exon5:c.G148A:p.G50R, |
| | ARAP1:NM_001135190:exon5:c.G148A:p.G50R, |
| 8 | BLK:NM_001715:exon4:c.G252C:p.K84N, |
| 9 | CACNA1A:NM_001127222:exon19:c.C2759G:p.A920G, |
| | CACNA1A:NM_001127221:exon19:c.C2762G:p.A921G, |
| 10 | CACNA1D:NM_001128839:exon30:c.C3854T:p.A1285V, |
| | CACNA1D:NM_001128840:exon30:c.C3854T:p.A1285V, |
| | CACNA1D:NM_000720:exon31:c.C3914T:p.A1305V, |
| 11 | CACNA1E:NM_001205293:exon20:c.C27131T:p.R911W, |
| | CACNA1E:NM_000721:exon20:c.C2731T:p.R911W, |
| | CACNA1E:NM_001205294:exon19:c.C2674T:p.R892W, |
| 12 | CACNA1H:NM_021098:exon10:c.C2030T:p.S677L, |
| | CACNA1H:NM_001005407:exon10:c.C2030T:p.S677L, |
| 13 | CAPN10:NM_023083:exon4:c.C598A:p.P200T, |
| | CAPN10:NM_023085:exon4:c.C598A:p.P200T, |
| 14 | CASR:NM_000388:exon4:c.G1192A:p.D398N, |
| | CASR:NM_001178065:exon4:c.G1192A:p.D398N, |
| 15 | CASR:NM_000388:exon7:c.G2824A:p.E942K, |
| | CASR:NM_001178065:exon7:c.G2854A:p.E952K, |
| 16 | CCKAR:NM_000730:exon2:c.A305G:p.N102S, |
| 17 | CD38:NM_001775:exon3:c.C418T:p.R140W, |
| 18 | DIS3L2:NM_152383:exon13:c.G1448A:p.R483Q, |
| 19 | DIS3L2:NM_152383:exon13:c.G1600A:p.G534R, |
| 20 | EIF2AK3:NM_004836:exon12:c.G2014A:p.E672K, |
| 21 | EPHA5:NM_004439:exon17:c.A2876G:p.H959R, |
| | EPHA5:NM_182472:exon16:c.A2810G:p.H937R, |
| 22 | FAM3D:NM_138805:exon5:c.C178T:p.P60S, |
| 23 | FASN:NM_004104:exon22:c.G3557T:p.C1186F, |
| 24 | FASN:NM_004104:exon24:c.C4127T:p.A1376V, |
| 25 | FASN:NM_004104:exon34:c.G5809A:p.V1937I, |
| 26 | FASN:NM_004104:exon42:c.C7301T:p.T2434I, |
| 27 | FASN:NM_004104:exon42:c.G7192T:p.A2398S, |
| 28 | FOXA2:NM_021784:exon2:c.G274A:p.A92T, |
| | FOXA2:NM_153675:exon3:c.G256A:p.A86T, |
| 29 | GAL:NM_015973:exon6:c.C368T:p.S123F, |
| 30 | GHSR:NM_198407:exon2:c.G1070A:p.R357Q, |
| 31 | GIPR:NM_000164:exon11:c.G947T:p.R316L, |
| 32 | GIPR:NM_000164:exon6:c.C406T:p.R136W, |
| 33 | GIPR:NM_000164:exon9:c.C843A:p.Y281X, |
| 34 | GLIS3:NM_152629:exon3:c.G951T:p.E317D, |
| | GLIS3:NM_001042413:exon4:c.G1416T:p.E472D, |
| 35 | GLIS3:NM_152629:exon7:c.G1819A:p.A607T, |
| | GLIS3:NM_001042413:exon8:c.G2284A:p.A762T, |
| 36 | GLIS3:NM_152629:exon8:c.A2006G:p.H669R, |
| | GLIS3:NM_001042413:exon9:c.A2471G:p.H824R, |
| 37 | GLP1R:NM_002062:exon2:c.G131A:p.R44H, |
| 38 | GPLD1:NM_001503:exon21:c.T2081C:p.M694T, |
| 39 | HLA-DRB5:NM_002125:exon2:c.C165A:p.F55L, |
| 40 | HNF4A:NM_001030003:exon2:c.C83T:p.A28V, |
| | HNF4A:NM_178850:exon2:c.C149T:p.A50V, |
| | HNF4A:NM_178849:exon2:c.C149T:p.A50V, |
| | HNF4A:NM_000457:exon2:c.C149T:p.A50V, |
| | HNF4A:NM_175914:exon2:c.C83T:p.A28V, |
| | HNF4A:NM_001030004:exon2:c.C83T:p.A28V, |
| 41 | HTT:NM_002111:exon12:c.G1652A:p.G551E, |
| 42 | INHBB:NM_002193:exon1:c.G157A:p.D53N, |
| 43 | IRS1:NM_005544:exon1:c.G2164A:p.G722S, |
| 44 | KANK1:NM_015158:exon3:c.C1991T:p.A664V, |
| | KANK1:NM_153186:exon2:c.C1517T:p.A506V, |
| 45 | KANK1:NM_015158:exon3:c.G1196A:p.R399Q, |
| | KANK1:NM_153186:exon2:c.G722A:p.R241Q, |
| 46 | KANK1:NM_015158:exon3:c.G1915A:p.V639I, |
| | KANK1:NM_153186:exon2:c.G1441A:p.V481I, |
| 47 | KCNB2:NM_004770:exon3:c.C2279A:p.T760N, |
| 48 | KCNH6:NM_030779:exon6:c.G1280A:p.R427Q, |
| 49 | KCNH6:NM_030779:exon6:c.G1354A:p.D452N, |
| 50 | KCNJ15:NM_170736:exon3:c.A88C:p.M30L, |
| | KCNJ15:NM_002243:exon4:c.A88C:p.M30L, |
| | KCNJ15:NM_170737:exon3:c.A88C:p.M30L, |
| 51 | KCNQ1:NM_000218:exon10:c.C1343G:p.P448R, |
| 52 | LBR:NM_002296:exon12:c.G1528A:p.A510T, |
| | LBR:NM_194442:exon12:c.G1528A:p.A510T, |
| 53 | LRP5:NM_002335:exon2:c.C290T:p.A97V, |
| 54 | LRP5:NM_002335:exon9:c.G1871A:p.R624Q, |
| 55 | MARCKS:NM_002356:exon2:c.C821T:p.A274V, |
| 56 | MC4R:NM_005912:exon1:c.G914A:p.R305Q, |
| 57 | MGEA5:NM_012215:exon1:c.G137A:p.G46E, |
| | MGEA5:NM_001142434:exon1:c.G137A:p.G46E, |
| 58 | MICU1:NM_001195519:exon3:c.T215G:p.L72R, |
| | MICU1:NM_001195518:exon8:c.T809G:p.L270R, |
| 59 | MYO5A:NM_000259:exon20:c.C2491T:p.R831C, |
| | MYO5A:NM_001142495:exon20:c.C2491T:p.R831C, |
| 60 | NNT:NM_182977:exon14:c.C1987T:p.L663F, |
| | NNT:NM_012343:exon14:c.C1987T:p.L663F, |
| 61 | NNT:NM_182977:exon3:c.A188G:p.K63R, |
| | NNT:NM_012343:exon3:c.A188G:p.K63R, |
| 62 | NOS2:NM_000625:exon19:c.A2239G:p.T747A, |
| 63 | NR0B2:NM_021969:exon1:c.C100T:p.R34X, |
| 64 | OXCT1:NM_000436:exon2:c.C173T:p.T58M, |
| 65 | PAM:NM_138822:exon19:c.T2153C:p.F718S, |
| | PAM:NM_001177306:exon19:c.T2153C:p.F718S, |
| | PAM:NM_000919:exon19:c.T2153C:p.F718S, |
| | PAM:NM_138766:exon19:c.T2153C:p.F718S, |
| | PAM:NM_138821:exon18:c.T1832C:p.F611S, |
| 66 | PAX4:NM_006193:exon1:c.G92A:p.R31Q, |
| 67 | PC:NM_001040716:exon15:c.A1702G:p.T568A, |
| | PC:NM_000920:exon14:c.A1702G:p.T568A, |
| | PC:NM_022172:exon13:c.A1702G:p.T568A, |
| 68 | PCK2:NM_004563:exon9:c.C1379T:p.P460L, |
| 69 | PDE4C:NM_001098818:exon1:c.C88T:p.L30F, |
| 70 | PDE8B:NM_001029853:exon18:c.C2084T:p.T695M, |
| | PDE8B:NM_003719:exon19:c.C2144T:p.T715M, |
| | PDE8B:NM_001029852:exon18:c.C1979T:p.T660M, |
| | PDE8B:NM_001029851:exon16:c.C1853T:p.T618M, |
| | PDE8B:NM_001029854:exon18:c.C2003T:p.T668M, |
| 71 | PRKCE:NM_005400:exon9:c.G1130C:p.R377P, |
| 72 | PSMD9:NM_002813:exon1:c.G31A:p.G11S, |
| 73 | RAPGEF3:NM_001098531:exon2:c.C79T:p.R27W, |
| 74 | REST:NM_001193508:exon4:c.A2241G:p.1747M, |
| | REST:NM_005612:exon4:c.A2241G:p.I747M, |
| 75 | RYR2:NM_001035:exon46:c.G7076A:p.R2359Q, |
| 76 | RYR2:NM_001035:exon56:c.A8419G:p.I2807V, |
| 77 | RYR3:NM_001243996:exon39:c.A6125C:p.N2042T, |
| | RYR3:NM_001036:exon39:c.A6125C:p.N2042T, |
| 78 | SCN9A:NM_002977:exon15:c.A2359G:p.M787V, |
| 79 | SIRT4:NM_012240:exon3:c.G703A:p.G235R, |
| 80 | SLC18A2:NM_003054:exon10:c.C899G:p.S300C, |
| 81 | SLC24A6:NM_024959:exon11:c.C1072T:p.L358F, |
| 82 | SLC2A2:NM_000340:exon3:c.G301A:p.V101I, |
| 83 | THADA:NM_022065:exon11:c.G1326T:p.E442D, |
| | THADA:NM_001083953:exon11:c.G1326T:p.E442D, |
| 84 | THADA:NM_022065:exon11:c.T1295A:p.V432D, |
| | THADA:NM_001083953:exon11:c.T1295A:p.V432D, |
| 85 | THADA:NM_022065:exon20:c.C2992G:p.R998G, |
| | THADA:NM_001083953:exon20:c.C2992G:p.R998G, |
| 86 | THADA:NM_022065:exon29:c.C4150G:p.R1384G, |
| | THADA:NM_001083953:exon29:c.C4150G:p.R1384G, |
| 87 | THADA:NM_022065:exon32:c.C4631G:p.S1544C, |
| | THADA:NM_001083953:exon32:c.C4631G:p.S1544C, |
| 88 | THADA:NM_022065:exon9:c.T776C:p.I259T, |
| | THADA:NM_001083953:exon9:c.T776C:p.I259T, |
| 89 | TRPM2:NM_003307:exon11:c.A1748G:p.N583S, |
| 90 | TRPM2:NM_003307:exon26:c.C3830T:p.T1277M, |
| 91 | TRPM4:NM_017636:exon4:c.C290T:p.T97M, |
| | TRPM4:NM_001195227:exon4:c.C290T:p.T97M, |
| 92 | TRPM5:NM_014555:exon5:c.G655C:p.G219R, |
| 93 | TRPV3:NM_145068:exon5:c.G443A:p.R148Q, |
| 94 | WFS1:NM_001145853:exon2:c.A41G:p.Q14R, |
| | WFS1:NM_006005:exon2:c.A41G:p.Q14R, |
| — | — |
| — | — |
| — | — |
| — | — |
| — | — |
| — | — |

### Experimental Example. Validation of SU Response by Suppression Gipr Gene Expression in Insulin-Secreting Cells

Among the 70 selected genes, three variants were observed in GIPR, specifically (Entry No. 31-33). Of these, Variant 33 (GIPR:NM_000164:exon9:c.C843A) is a stop-gained variant that generates a stop codon, terminating translation at the 281st position of the 462-amino acid GIPR peptide (p.Y281X). Such a premature stop codon is well-known to suppress the mRNA transcription of the gene through a mechanism called nonsense-mediated decay (NMD) (J Biol Chem. 2022 November; 298(11):102592). Thus, Variant 33 has a theoretical basis for suppressing GIPR expression. Another GIPR variant, Entry No. 31, was also predicted to affect protein function based on in silico functional predictions using Polyphen and SIFT (Table 4).

TABLE 4

| | | | | | | MAF | MAF | |
|---|---|---|---|---|---|---|---|---|
| | Entry number | | Position | | | Global | EAS | predicted |
| Gene | in table 3 | rsID | (GRCh37/hg19) | Reference | Alternate | (×10⁻³) | (×10⁻³) | effect |
| GIPR | 31 | rs13306403 | 19-46181186 | G | T | 0.064 | 0.815 | deleterious* |
| | 32 | rs13306402 | 19-46177353 | C | T | 0.883 | 9.22 | tolerable* |
| | 33 | rs550405192 | 19-46180630 | C | A | 0.063 | 0.833 | stop gained |

EAS: East Asian;
MAF: Minor allele frequency
*in silico prediction by Polyphen and SIFT

Furthermore, GIPR protein is a cell membrane receptor that transmits signals from the incretin hormone GIP (gastric inhibitory polypeptide). Diabetes medications based on the incretin effect, such as DPP-4 inhibitors, have been reported to interact with SU, inducing hypoglycemia. It was hypothesized that suppression of GIPR expression may influence SU responsiveness in pancreatic beta cells. Therefore, the relationship between GIPR expression suppression and SU dependency was validated through in vitro experiments.

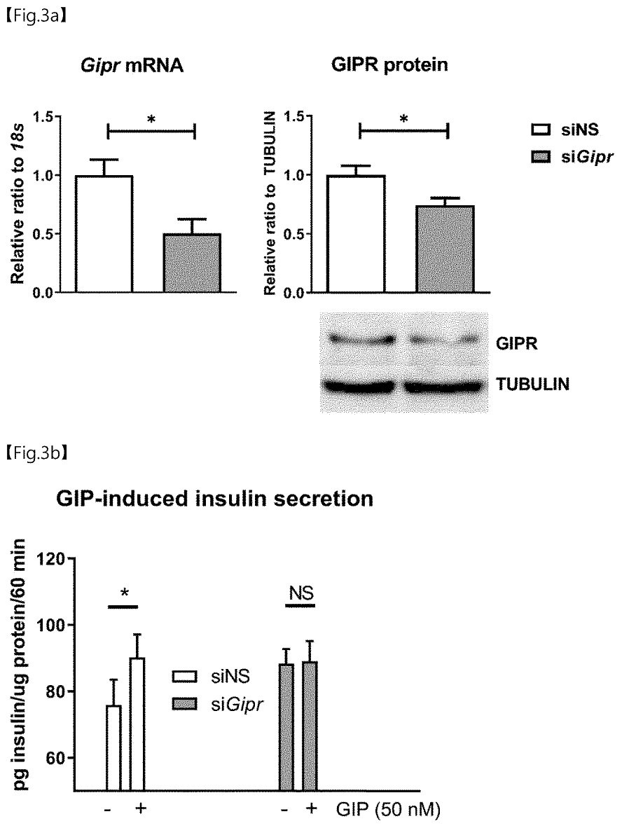

Experiments were conducted to suppress the expression of the rat Gipr gene, which shares the same gene sequence as human GIPR. Gipr mRNA and protein expressions were significantly suppressed in rat insulinoma cells using siRNA (FIG. 3a), which functionally inhibited GIP-induced insulin secretion (FIG. 3b).

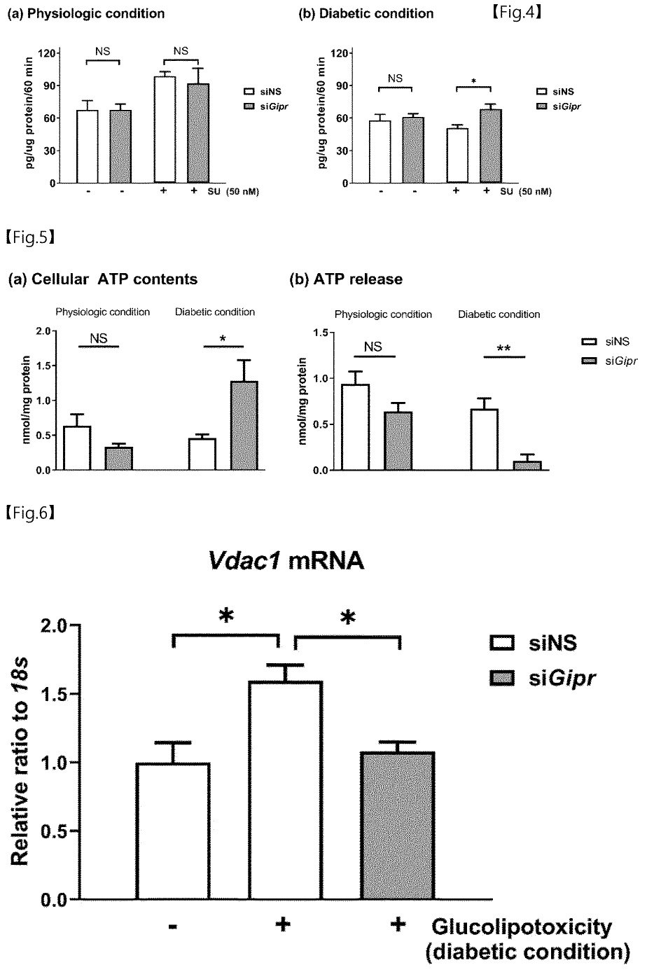

Next, the SU response was examined based on Gipr suppression. Under physiological conditions, Gipr suppression did not affect insulin secretion in response to SU stimulation (FIG. 4a). However, under diabetic conditions—chronic exposure to high glucose and fatty acids (glucolipotoxicity)—Gipr suppression increased insulin secretion in response to low-dose SU (FIG. 4b).

Since SU-induced insulin secretion depends on intracellular ATP, the effect of Gipr suppression on ATP was evaluated. Under physiological conditions, Gipr suppression did not alter ATP content. However, under diabetic conditions, intracellular ATP levels increased (FIG. 5a). Conversely, extracellular ATP release was significantly reduced under diabetic conditions when Gipr expression was suppressed (FIG. 5b). These results suggest that Gipr suppression under diabetic conditions inhibits ATP release, increases intracellular ATP levels, and enhances SU response.

Additionally, in insulin-secreting cells, it has been reported that intracellular ATP levels are regulated by voltage-dependent anion channel 1 (VDAC1) (Cell Metab. 2019 Jan. 8; 29(1):64). Specifically, under normal conditions, VDAC1 is located on the outer mitochondrial membrane. However, under diabetic conditions, VDAC1 expression increases, translocates to the cell membrane, promotes extracellular ATP release, and ultimately suppresses insulin secretion. Analysis of Vdac1 gene expression in rat insulinoma cells revealed that Vdac1 expression increased under diabetic conditions, while Gipr suppression inhibited this increase (FIG. 6).

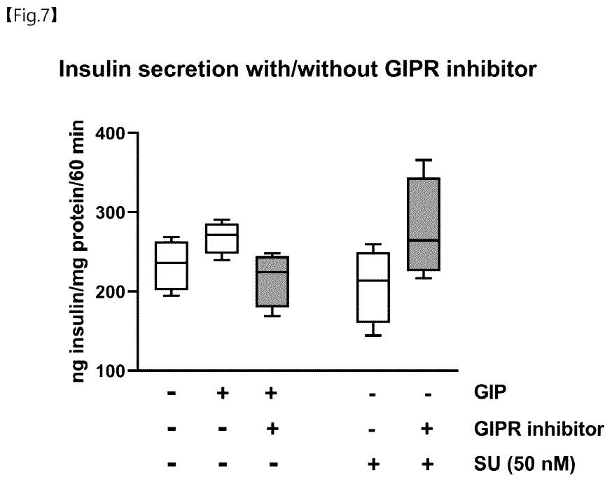

Furthermore, treatment with a GIPR inhibitor (rat GIP(3-30), 100 nM, 24 h), which directly inhibits the function of GIPR protein rather than its gene expression, showed a trend of enhanced insulin secretion by SU even under non-diabetic conditions (FIG. 7).

In summary, GIPR suppression, both in terms of expression and function (FIGS. 3 and 7), inhibited glucolipotoxicity-induced VDAC1 expression (FIG. 6), in turn, blocked extracellular ATP release (FIG. 5b), increased intracellular ATP content (FIG. 5a), and contributed to enhanced SU-induced insulin secretion (FIGS. 4 and 7).

Collectively, as shown in FIG. 1, candidate variants were identified from clinically distinct patients (Table 3). Among these, Variant 33 of GIPR is likely to suppress transcription via NMD. Based on this, experiments suppressing GIPR transcription or function in cell lines confirmed enhanced insulin secretion in response to SU. Therefore, other SNVs in GIPR that potentially suppress transcription via NMD are also predicted to increase the response to SU (GIPR stop-gain variants: Source: gnomAD v2.1.1. https://gnomad.broadinstitute.org/) (Table 1).

The pharmacogenetics of T2DM is not currently applied in clinical practice. However, it holds potential for application in subgroups such as SU-dependent patients. This study identified novel single nucleotide variants (SNVs) associated with SU responsiveness in type 2 diabetes. These findings could contribute to predicting SU responses through genetic analysis.

1. A composition for predicting sulfonylurea dependency in diabetes mellitus patients, comprising an agent that identifies the mRNA level of GIPR (gastric inhibitory polypeptide receptor), the level or activity of GIPR protein, or mutations inhibiting the level or activity of GIPR.

2. The composition for predicting sulfonylurea dependency in diabetes mellitus patients according to claim 1, wherein the mutations are missense mutations, frameshift mutations, nonsense mutations, or splice site mutations.

3. The composition for predicting sulfonylurea dependency in diabetes mellitus patients according to claim 1, wherein the agent is a primer or probe that specifically binds to the GIPR gene.

4. The composition for predicting sulfonylurea dependency in diabetes mellitus patients according to claim 1, wherein the mutations are one or more single nucleotide variations (SNVs) selected from the group consisting of rs550405192, rs13306403, rs13306402, rs554179666, rs1194979043, rs149510000, rs759654048, rs764005735, rs935395843, rs755629061, rs749728382, rs779198689, rs1271638992, rs775963892, rs778756249, rs146268621, rs753645152, rs771165150, rs771830344, rs144328094, rs1159478274, rs1292381802, rs550405192, rs747395645, rs1183524419, and rs757704281.

5. A composition for modulating sulfonylurea dependency in diabetes mellitus patients, comprising an agent that regulates the mRNA level of GIPR (gastric inhibitory polypeptide receptor), the level or activity of GIPR protein, or induces mutations that inhibit the level or activity of GIPR.

6. The composition for modulating sulfonylurea dependency in diabetes mellitus patients according to claim 5, wherein the mutations in GIPR are one or more single nucleotide variations (SNVs) selected from the group consisting of rs550405192, rs13306403, rs13306402, rs554179666, rs1194979043, rs149510000, rs759654048, rs764005735, rs935395843, rs755629061, rs749728382, rs779198689, rs1271638992, rs775963892, rs778756249, rs146268621, rs753645152, rs771165150, rs771830344, rs144328094, rs1159478274, rs1292381802, rs550405192, rs747395645, rs1183524419, and rs757704281.

7. A method for providing information to predict sulfonylurea dependency in diabetes mellitus patients, comprising the step of identifying the mRNA level of GIPR (gastric inhibitory polypeptide receptor), the level or activity of GIPR protein, or mutations in GIPR from a biological sample isolated from an individual.

8. The method for providing information to predict sulfonylurea dependency in diabetes mellitus patients according to claim 7, wherein the mutations are missense mutations, frameshift mutations, nonsense mutations, or splice site mutations.

9. The method for providing information to predict sulfonylurea dependency in diabetes mellitus patients according to claim 7, wherein the agent is a primer or probe that specifically binds to the GIPR gene.

10. The method for providing information to predict sulfonylurea dependency in diabetes mellitus patients according to claim 7, wherein the mutations in GIPR are one or more single nucleotide variations (SNVs) selected from the group consisting of rs550405192, rs13306403, rs13306402, rs554179666, rs1194979043, rs149510000, rs759654048, rs764005735, rs935395843, rs755629061, rs749728382, rs779198689, rs1271638992, rs775963892, rs778756249, rs146268621, rs753645152, rs771165150, rs771830344, rs144328094, rs1159478274, rs1292381802, rs550405192, rs747395645, rs1183524419, and rs757704281.

11. The method for providing information to predict sulfonylurea dependency in diabetes mellitus patients according to claim 7, further comprising the step of providing information that an individual with the mutations has sulfonylurea dependency.

12. The method for providing information to predict sulfonylurea dependency in diabetes mellitus patients according to claim 7, further comprising the step of providing information that if the level or activity is lower than that of a control group, the individual is more likely to have sulfonylurea dependency compared to the control group.


SEQUENCE LISTING (2 sequences)

SEQ ID NO: 1 - RNA, 2025 nt, Homo sapiens
  source 1..2025: mol_type = mRNA
ggcagcggtggcaggggctgcaggagcaagtgaccaggagcaggactggggacaggcctgatcgcccctgcacgaaccagacccttcgccgccctcacgatgactacctctccgatcctgcagctgctgctgcggctctcactgtgcgggctgctgctccagagggcggagacaggctctaaggggcagacggcgggggagctgtaccagcgctgggaacggtaccgcagggagtgccaggagaccttggcagccgcggaaccgccttcaggcctcgcctgtaacgggtccttcgatatgtacgtctgctgggactatgctgcacccaatgccactgcccgtgcgtcctgcccctggtacctgccctggcaccaccatgtggctgcaggtttcgtcctccgccagtgtggcagtgatggccaatggggactttggagagaccatacacaatgtgagaacccagagaagaatgaggcctttctggaccaaaggctcatcttggagcggttgcaggtcatgtacactgtcggctactccctgtctctcgccacactgctgctagccctgctcatcttgagtttgttcaggcggctacattgcactagaaactatatccacatcaacctgttcacgtctttcatgctgcgagctgcggccattctcagccgagaccgtctgctacctcgacctggcccctaccttggggaccaggcccttgcgctgtggaaccaggccctcgctgcctgccgcacggcccagatcgtgacccagtactgcgtgggtgccaactacacgtggctgctggtggagggcgtctacctgcacagtctcctggtgctcgtgggaggctccgaggagggccacttccgctactacctgctcctcggctggggggcccccgcgcttttcgtcattccctgggtgatcgtcaggtacctgtacgagaacacgcagtgctgggagcgcaacgaagtcaaggccatttggtggattatacggacccccatcctcatgaccatcttgattaatttcctcatttttatccgcattcttggcattctcctgtccaagctgaggacacggcaaatgcgctgccgggattaccggctgaggctggctcgctccacgctgacgctggtgcccctgctgggtgtccacgaggtggtgtttgctcccgtgacagaggaacaggcccggggcgccctgcgcttcgccaagctcggctttgagatcttcctcagctccttccagggcttcctggtcagcgtcctctactgcttcatcaacaaggaggtgcagtcggagatccgccgtggctggcaccactgccgcctgcgccgcagcctgggcgaggagcaacgccagctcccggagcgcgccttccgggccctgccctccggctccggcccgggcgaggtccccaccagccgcggcttgtcctcggggaccctcccagggcctgggaatgaggccagccgggagttggaaagttactgctagggggcgggatccccgtgtctgttcagttagcatggatttattgagtgccaactgcgtgccaggcccagtacggaggacgctggggaaatggtgaaggaaacagaaaaaaggtccctgcccttctggagatgacaactgagtggggaaaacagaccgtgaacacaaaacatcaagttccacacacgctatggaatggttatgaagggaagcgagaagggggcctagggtggtctgggaggcgtctccaaggaggtgacacttaagccatccccgaaagaggtgaaagagatcactttggggagagctggagaacaggattctaggcggaagcgatagcataggcaaaggcccttgggcaggaaggcgctcagccttggctggagtagaattaagtcagagccaacaggttggggagagacagagaagtgggcaggggcacccaagttgggatttcatttcaggtgcattggagattcttaggagtgtctcttgggggtaatattttattttttaaaaaatgaggat

SEQ ID NO: 2 - protein, 430 aa, Homo sapiens
MTTSPILQLLLRLSLCGLLLQRAETGSKGQTAGELYQRWERYRRECQETLAAAEPPSVAAGFVLRQCGSDGQWGLWRDHTQCENPEKNEAFLDQRLILERLQVMYTVGYSLSLATLLLALLILSLFRRLHCTRNYIHINLFTSFMLRAAAILSRDRLLPRPGPYLGDQALALWNQALAACRTAQIVTQYCVGANYTWLLVEGVYLHSLLVLVGGSEEGHFRYYLLLGWGAPALFVIPWVIVRYLYENTQCWERNEVKAIWWIIRTPILMTILINFLIFIRILGILLSKLRTRQMRCRDYRLRLARSTLTLVPLLGVHEVVFAPVTEEQARGALRFAKLGFEIFLSSFQGFLVSVLYCFINKEVQSEIRRGWHHCRLRRSLGEEQRQLPERAFRALPSGSGPGEVPTSRGLSSGTLPGPGNEASRELESYC